(19) United States
(12) Patent Application Publication
(10) Pub. No.: US 2026/0174826 A1
(43) Pub. Date: Jun. 25, 2026

(54) HYDROLYZED COLLAGEN COMPOSITIONS AND METHODS OF MAKING THE SAME

(71) Applicant: ROCHAL TECHNOLOGIES, LLC, San Antonio, TX (US)

(72) Inventors: Ron T. NIXON, Houston, TX (US); Ann Beal SALAMONE, Gainesville, FL (US); Nina BIONDA, Ithaca, NY (US); Rebecca E. McMAHON, San Antonio, TX (US)

(73) Assignee: ROCHAL TECHNOLOGIES, LLC, San Antonio, TX (US)

(21) Appl. No.: 19/418,476

(22) Filed: Dec. 12, 2025

Related U.S. Application Data
(60) Provisional application No. 63/736,196, filed on Dec. 19, 2024.

(51) Int. Cl. A61K 38/01; A61K 31/14; A61K 31/17; A61K 31/496; A61K 31/65; A61K 31/7036; A61K 33/18; A61K 33/34; A61K 33/38; A61K 38/12; A61K 45/06; A61K 47/36; A61K 47/46

(52) U.S. Cl. CPC A61K 38/014; A61K 31/14; A61K 31/17; A61K 31/496; A61K 31/65; A61K 31/7036; A61K 33/18; A61K 33/34; A61K 33/38; A61K 38/12; A61K 45/06; A61K 47/36; A61K 47/46

(57) ABSTRACT

Hydrolyzed collagen compositions are described that are used for treatment of impaired tissue including mucosal tissue or to prevent damage to healthy tissue. These compositions function to treat mucosal tissues found in the digestive, urinary, respiratory and reproductive systems. Oral or topical, site-specific delivery of the compositions may be via liquid, spray, gel, powder, tablet, capsule, or sheet form.

### CROSS-REFERENCE TO RELATED APPLICATIONS

This application claims priority under 35 U.S.C. § 119(e) to U.S. Provisional Application No. 63/736,196, filed on Dec. 19, 2024, the entire content of which is incorporated herein by reference.

### FIELD OF INVENTION

This invention relates generally to compositions for treatment of impaired tissue, including mucosal tissues, and for prevention of tissue damage.

### BACKGROUND

Mucosa functions to retain tissue moisture and prevent dehydration, to prevent pathogens and particulates from entering the body, to serve as an interface between animal tissue and microbiome, to protect the body from itself (i.e., gastric juices, urine), to absorb and transform nutrients and medications, and to participate in immune system responses. As used herein, “mucosa” has its standard meaning and includes transitional epithelium and lamina propria, but not submucosa.

Digestive system mucosal tissue lines the mouth, esophagus, stomach, intestines, and anus. Mucosal tissue in the urinary tract lines the renal pelvis, bladder, ureters, and urethra. The nasal cavity, trachea, and bronchial tubes are also lined with mucosal tissue.

Mucosal tissue is composed of two principal layers: (1) apical layer(s) are composed of epithelial cells and keratin and are where mucus is secreted to provide hydration and barrier function; and (2) the connecting layer (lamina propria) that attaches the mucus-secreting apical layer(s) to the body and which delivers nutrients from the blood stream to the epithelial cells. Mucus (mucin: mucopolysaccharides, glycoproteins, and salts) hydrates the mucosal tissue and is a first line of defense against pathogenic microorganisms, toxins, and acute trauma. Mucus is a lubricant, which minimizes shear stress to the underlying tissue from food, air, excrement, and reproductive movement. Mucins aid in fibroblast proliferation, keratinocyte turnover, and growth factor release (Toma 2021). Intraepithelial T cells as well as lymphoid cells are found between epithelial cells in the mucosa and regulate inflammation, immune-response and homeostasis. Mucosal pH of 5.5 to 7 provides homeostasis as well as wound healing.

Mucosal protection from exogeneous microorganisms and toxins occurs through physical barrier function as well as immune system response. The mucosa biochemically provides protection through regulatory T cells (Tregs) with 75% of all lymphocytes in the human body being present in the mucosa (Brandtzaeg 2009).

Traumatic injury and surgical damage to mucosal tissue leads to a similar healing process as with dermal wounds—hemostasis, inflammation, cellular and extracellular repair, and remodeling. A variety of polymeric scaffolds, including autologous mucosal tissue, are utilized to improve mucosal trauma-induced wound healing.

However, when mucosal tissue is inflamed with insufficient inflammation control, mucositis occurs with erythema, edema and ulcerations of the mucosal tissue. It is a common outcome from treatments such as radiation therapy, chemotherapy, chemoradiation therapy, as well as myeloablative preparations for hematopoietic stem cell transplantation. Oral mucositis is debilitating and painful, with severe cases resulting in the need for parenteral feeding. With the decrease in the protective function of the mucosal barrier due to inflammation and resulting cell death (mucositis), infection occurs both locally and systemically, depending on the affected area.

Radiation-induced oral mucositis occurs with 100% of head and neck cancer patients treated with altered fractionation radiotherapy (Bell 2023). Patients (20-40%) receiving chemotherapy for solid tumors frequently develop mucositis within five to fourteen days of starting treatment (Bell 2023). These treatments adversely affect epithelial cells by delivering upregulated inflammatory proteins through the capillaries present in the mucosal lamina.

Clinical treatment for oral mucositis consists of infection control, pain management, inflammation control and lubrication of the mucosa. One means of infection control includes frequent brushing, and use of oral rinses (saline, sodium bicarbonate, mixtures of both). For pain management, morphine 0.2% mouth rinse is frequently used. Another clinically used mouth rinse contains lidocaine, diphenhydramine, and magnesium aluminum hydroxide. During short-term chemotherapy treatment, cold water or ice is applied to the mucosal to decrease blood flow and, hence, decrease chemotherapy agent access to the mucosa. A non-steroidal anti-inflammatory drug (NSAID), benzydamine, is used as a spray to decrease inflammation and increase new capillary proliferation. A keratinocyte growth factor, Palifermin, may be used for severe oral mucositis caused by myeloablative treatment. Zinc supplementation in the diet is used to promote mucosal healing. Lubrication of the mucosa can be provided by mucoadhesive coatings that retain moisture and have a low coefficient of friction. These treatments mitigate mucositis to some extent.

### SUMMARY OF THE INVENTION

In one aspect, a composition comprising hydrolyzed collagen and low molecular weight polysaccharides is provided.

In some embodiments, a hydrolyzed collagen-containing composition described herein comprises hydrolyzed collagen and low molecular weight polysaccharides. In some embodiments, the hydrolyzed collage-containing composition comprises 25 wt % to 99.99 wt % of hydrolyzed collagen and 0.01 to 50 wt % of low molecular weight polysaccharides, wherein percentages are based on the percent solids of the composition.

The hydrolyzed collagen-containing composition can have anti-inflammatory properties to mitigate tissue degradation resulting from overexpression of inflammatory proteins and reactive ion species.

It is an object of the hydrolyzed collagen-containing compositions described herein to improve cell migration and proliferation in mammalian tissue.

It is an object of the hydrolyzed collagen-containing compositions described herein to facilitate tissue healing.

It is an object of the hydrolyzed collagen-containing compositions described herein where the compositions can be used in mucosa hygiene products.

It is an object of the hydrolyzed collagen-containing compositions described herein to treat surgical sites.

It is an object of the hydrolyzed collagen-containing compositions described herein to treat acute and chronic wounds, as well as burn wounds.

It is an object of the hydrolyzed collagen-containing compositions described herein to treat tissue impacted by radiation therapy, chemotherapy, and combinations thereof.

It is an object of the hydrolyzed collagen-containing compositions described herein to treat tissue impacted by myeloablative therapy.

It is an object of the hydrolyzed collagen-containing compositions described herein where the composition can include an antimicrobial agent.

It is an object of the hydrolyzed collagen-containing compositions described herein where the composition comprises an antimicrobial agent which compositions are not cytotoxic to mucosal cells.

It is an object of the hydrolyzed collagen-containing compositions described herein to reduce and eliminate Gram-positive and Gram-negative bacteria in wounds, tissues, surfaces and devices.

It is an object of the hydrolyzed collagen-containing compositions described herein to reduce and eliminate fungi in wounds, tissues, surfaces and devices.

It is an object of the hydrolyzed collagen-containing compositions described herein to reduce and eliminate yeast in wounds, tissues, surfaces and devices.

It is an object of the hydrolyzed collagen-containing compositions described herein to reduce and eliminate viruses in wounds, tissues, surfaces and devices.

It is an object of the hydrolyzed collagen-containing compositions described herein where the composition can comprise a mucoadhesive which can be cationic, ionic, amphoteric or neutral in charge.

It is an object of the hydrolyzed collagen-containing compositions described herein where the composition can comprise a cationic mucoadhesive to provide substantivity to mucosal tissue.

It is an object of the hydrolyzed collagen-containing compositions described herein where the composition can be deposited on a tissue surface or a device in contact with mucosa.

It is an object of the hydrolyzed collagen-containing compositions described herein to treat mucosal tissue according to any of the foregoing objects.

These and other objectives and advantages of the hydrolyzed collagen-containing compositions described herein, some of which are specifically described and others that are not, will become apparent from the detailed description.

### DETAILED DESCRIPTION OF THE INVENTION

In this specification and the appended claims that follow, reference will be made to a number of terms that shall be defined to have the following meanings:

It must be noted that, as used in the specification and the appended claims, the singular forms “a”, “an” and “the” include plural references unless the context clearly dictates otherwise. Thus, for example, reference to “a mucoadhesive” includes mixtures of two or more such mucoadhesives, and the like.

As used herein, the term “about” indicates that a value can vary by up to ±5%, ±2%, or ±1%.

In one aspect, a composition comprising hydrolyzed collagen and low molecular weight polysaccharides is provided. As used herein, “hydrolyzed collagen-containing composition” and “composition” can be used interchangeably to reference the compositions described herein.

In some embodiments, a hydrolyzed collagen-containing composition is provided that includes both hydrolyzed collagen and low molecular weight polysaccharides. In some embodiments, a hydrolyzed collagen-containing composition includes hydrolyzed collagen and 0.01 wt % to 50 wt % low molecular weight polysaccharides based on the percent solids of the hydrolyzed collagen-containing composition. As used herein, “percent solids” has its standard meaning of the quantification of the non-volatile ingredients in a composition.

In some embodiments, the hydrolyzed collagen-containing composition comprises 25 wt % to 99.99 wt-% of hydrolyzed collagen. In some embodiments, the hydrolyzed collagen-containing composition comprises up to 99.9 wt-% or up to 99 wt-% of hydrolyzed collagen based on the percentage of solids in the composition. In some embodiments, the hydrolyzed collagen-containing composition comprises at least 30 wt-%, at least 40 wt-%, at least 50 wt-%, at least 60 wt-%, at least 80 wt-%, or at least 90 wt-%, or at least 95 wt-% of hydrolyzed collagen based on the percentage of solids in the composition.

In some embodiments, the source of collagen used to form the hydrolyzed collagen can be human, bovine, porcine, piscine, ovine, avian, or multiple sources. In some embodiments, the hydrolyzed collagen is derived from bovine collagen.

In some embodiments, the hydrolyzed collagen has a molecular weight of up to 12,000 Da or up to 10,000 Da. In some embodiments, the hydrolyzed collagen has a molecular weight of at least 75 Da.

In some embodiments, the hydrolyzed collagen-containing composition comprises 0.01 wt % to 50 wt % of low molecular weight polysaccharides based on the percent solids of the composition.

In some embodiments, the hydrolyzed collagen-containing composition comprises 0.1 wt % to 1 wt % or 1 to 15 wt-% of low molecular weight polysaccharides based on the percent solids of the composition. In some embodiments, the hydrolyzed collagen-containing compositions comprises up to 20 wt %, or 10 wt %, or 5 wt %, or 2 wt % low molecular weight polysaccharides based on the percent solids of the composition.

The low molecular weight polysaccharides may be present naturally or can be added to or blended with the hydrolyzed collagen. In some embodiments, the low molecular weight polysaccharides can have a molecular weight range from 400 to 12,000 Da. In some embodiments, the low molecular weight polysaccharides can have a molecular weight range from 400 to 600 Da, 600 to 11,000 Da, or from 1,000 to 10,000 Da.

In some embodiments, the low molecular weight polysaccharides comprise oligosaccharides, low molecular weight polysaccharides, or a combination thereof. The low molecular weight polysaccharides may be derived from microbial, plant and/or animal sources. In some embodiments, the low molecular weight polysaccharides can be homopolysaccharides (e.g., low molecular weight starch, glycogen, cellulose) or heteropolysaccharides (e.g., low molecular weight arabinoxylans, glucomannans, carrageenans, fucoidans), which can be linear or branched.

In some embodiments, the low molecular weight polysaccharides include one or more monosaccharides, such as, glucose, mannose, arabinose, rhamnose, galactose, fructose, xylose, iduronic acid, mannuronic acid, glucuronic acid, as well as, amino sugars, such as N-acetyl-D-glucosamine.

In some embodiments, low molecular weight polysaccharides can be derived through chemical, physical or enzymatic degradation processes from higher molecular weight polysaccharides. In some embodiments, the higher molecular weight polysaccharides can be selected from sources including, but not limited to, β-glucan, xylans, hyaluronic acid, chitosan, xanthan gum, gellan gum, scleroglucans, carrageenan, ulvan, alginate, fucoidan, and glucomannans.

In some embodiments, the higher molecular weight polysaccharides can be derived from sources including, but not limited to, glycoproteins, proteoglycans, glycosaminoglycans and combinations thereof. In some embodiments, the higher molecular weight polysaccharides can be degradations products of sources including, but not limited to, glycoproteins, proteoglycans, glycosaminoglycans and combinations thereof. In some embodiments, the degradation can be one or more of chemical, physical, or enzymatic degradation

In some embodiments, the low molecular weight polysaccharides are derived from degradation of ulvans, fucoidans, and xylans.

In some embodiments, the hydrolyzed collagen-containing composition described herein synergistically increases cellular proliferation, mobility, and/or viability while decreasing inflammation when compared to either hydrolyzed collagen alone or low molecular weight polysaccharide alone.

Further, the hydrolyzed collagen-containing composition described herein rapidly spreads and absorbs onto and into mammalian tissue, mucus and mucosal tissue and, hence, is available for repair and healing of the underlying tissue.

In some embodiments, the hydrolyzed collagen-containing composition further includes at least one antimicrobial to hinder proliferation of microorganisms. In some embodiments, the addition of an antimicrobial agent helps to reduce or eliminate undesirable microbial colonies and biofilm formation. The compositions described herein can include an antimicrobial in an amount sufficient to hinder or eradicate microorganisms, in order to reduce the possibility of infection in the damaged tissue being treated.

In some embodiments, the antimicrobial agents can include, but are not limited to, polylysines, biguanides, such as poly(hexamethylene biguanide) (PHMB) and its salts, a low molecular weight synthetic cationic biguanide polymer, chlorhexidine and its salts, such as chlorhexidine digluconate, and alexidine and its salts, such as alexidine dihydrochloride, where the latter two are bis(biguanides), benzalkonium chloride, benzethonium chloride, cetyltrimethylammonium bromide, glycerol mono-laurate, capryl glycol, gentamicin sulfate, iodine, povidone iodine, starch-iodine, neomycin sulfate, polymyxin B, bacitracin, tetracyclines, clindamycin, gentamicin, nitrofurazone, mafenide acetate, copper and its salts, silver nanoparticles, silver sulfadiazine, silver nitrate, terbinafine hydrochloride, miconazole nitrate, ketoconazole, clotrimazole, itraconazole, metronidazole, antimicrobial peptides, polyquaternium-1, polyquatemium-6, polyquaternium-10, salts thereof, and combinations thereof.

In some embodiments, the antimicrobial agent comprises a biguanide selected from poly(hexamethylene biguanide) (PHMB) and its salts. In some embodiments, the antimicrobial agent comprises chlorhexidine digluconate. In some embodiments, the antimicrobial agent comprises alexidine dihydrochloride.

In some embodiments, the antimicrobial agent is or includes polylysine.

In some embodiments, the hydrolyzed collagen-containing composition described herein can include the antimicrobial agent at a concentration ranging from 0.0001 wt % to 1 wt % based on the total solids of the composition. In some embodiments, the hydrolyzed collagen-containing composition described herein can include the antimicrobial agent at a concentration ranging from 0.01 wt % to 0.75 wt %, or from 0.05 wt % to 0.5 wt %, or from 0.1 wt % to 0.25 wt % based on the total solids of the composition.

In some embodiments, the hydrolyzed collagen-containing composition can be a dry composition that includes the antimicrobial agent at a concentration ranging from 0.002 wt % to 25.0 wt %, or ranging from 0.20 wt % to 15.0 wt %, or ranging from 1.0 wt % to 10.0 wt %, or ranging from 2.0 wt % to 4.0 wt %, based on the total weight of the composition (e.g., dry).

In some embodiments, the hydrolyzed collagen-containing composition further comprises at least one mucoadhesive.

Hydrolyzed collagen and low molecular weight polysaccharides dissolve quickly in water without forming a hydrogel. In addition, they contain low molecular weight peptides and polysaccharides that downregulate inflammatory markers such as those produced during radiation and chemotherapies as well as upregulate cell proliferation and migration. Therefore, a mucoadhesive that can form a gel or a coating on mucosal tissue or mucin can provide substantivity and sustained delivery of the hydrolyzed collagen-containing composition.

Mucoadhesives can adhere to mucus or mucosal epithelial layers. Absorption of water serves to plasticize mucoadhesives and interpenetration occurs into the mucus through van der Waals forces and hydrogen bonds. Further, as mucoadhesives absorb water, they can swell providing physical entrenchment into the mucus and mucosal epithelial layer; hence, enabling diffusion and cohesive mechanisms for delivery of the composition's ingredients. When mucoadhesives are positively charged, they are ionically attracted to the negatively charged mucin/mucus. Utilizing similar surface free energies between mucin and the mucoadhesive increases contact area and wettability with higher surface energies for both mucin and mucuoadhesive, which increases the adhesive strength between them.

In some embodiments, mucoadhesives include at least one of chitosan, chitosan/8-glycerophosphate (a thermosensitive gel), thiolated-chitosan, Eudragits (poly(dimethylaminoethyl methacrylate-co-butyl methacrylate-co-methyl methacrylate), gelatin, polymethylmethacrylate, gellan gum, silica particles, poly(amidoamine), poly(N-vinyl-2-pyrrolidone), poly(vinyl alcohol), poly(2-hydroxyethyl methacrylate), Pluronics and Poloxamers (polyethylene oxide block copolymers with polypropylene oxide) (PEO-PPO-PEO), Carbopols (acrylic acid-based polymers), sodium alginate, hydroxypropyl cellulose, carboxymethylcellulose, alginates, pectins, high molecular weight polysaccharides, or Polymer JR (hydroxyethylcellulose reacted with trimethylammonium substituted epoxide).

In some embodiments, the hydrolyzed collagen-containing composition can comprise cationic mucoadhesives such as Dow UCARE Polymer JR-30M, Polymer JR-400, Polymer JR-125 and Eudragit E100.

In some embodiments, the hydrolyzed collagen-containing composition described herein can include a mucoadhesive at a concentration ranging from 0.01 wt % to 0.05 wt % or ranging from 0.1 wt % to 0.25 wt % based on the total solids of the composition. In some embodiments, dry hydrolyzed collagen-containing compositions described herein can include a mucoadhesive at a concentration ranging from 0.01 wt % to 25.0 wt %, or ranging from 0.20 wt % to 15.0 wt %, or ranging from 1.0 wt % to 10.0 wt %, or ranging from 2.0 wt % to 4.0 wt %, based on the total solids of the composition.

In some embodiments the hydrolyzed collagen-containing composition can include a water-soluble polymer to increase solution viscosity and to prolong residence time on or in the mucosa. In some embodiments, the composition can be an aqueous-based solution, gel, paste, emulsion, spray, or foam. In some embodiments, the composition can be in a dry form intended to form an aqueous-based solution, gel, paste, emulsion, spray, or foam when hydrated by aqueous media (e.g., water, saline, blood, plasma, mucous, etc.) when hydrated. In some embodiments, useful water-soluble polymers include, but are not limited to, poly(ethylene glycol), poly(ethylene oxide), poly(vinyl alcohol) and copolymers, poly(N-vinyl-2-pyrrolidone) and copolymers, methylcellulose, hydroxyethylcellulose, hydroxypropylcellulose, hydroxypropylmethylcellulose, carboxymethylcellulose, guar gum, hydroxyethylguar, hydroxypropylguar, gelatin, albumin, hydroxypropylmethylguar, carboxymethylguar, carboxymethylchitosan, locust bean gum, carrageenan, xanthan gum, gellan gum, pullulan, alginate, chondroitin sulfate, dextran, dextran sulfate, Aloe vera gel, scleroglucan, schizophyllan, gum arabic, tamarind gum, poly(methyl vinyl ether), ethylene oxide-propylene oxide-ethylene oxide block copolymers, hyaluronan, chondroitin sulfate, keratan sulfate, dermatan sulfate, heparan sulfate, dextran, carbomer and its salts, poly(acrylic acid) and its salts, poly(methacrylic acid) and its salts, poly(ethylene-co-acrylic acid), poly(vinyl methyl ether), poly(vinylphosphoric acid) salts, poly(vinylsulfonic acid) salts, sodium poly(2-acrylamido-2-methylpropanesulfonate), polyacrylamide(s), poly(N,N′-dimethylacrylamide), poly(N-vinylacetamide), poly(N-vinylformamide), poly(2-hydroxyethyl methacrylate), poly(glyceryl methacrylate), poly(2-ethyl-2-oxazoline), poly(N-isopropylacrylamide) and poly(N-vinylcaprolactam), the latter two hydrated below their Lower Critical Solution Temperatures, polyquaternium-1, polyquaternium-6, poly-quaternium-10, ionene polymers, cationic guar, pyridinium polymers, imidazolium polymers, diallyldimethylammonium polymers, acryloyl-, methacryloyl-, and styryl-trimethylammonium polymers, acrylamido- and methacrylamido-trimethylammonium polymers, and the like, and derivatives and combinations thereof.

In some embodiments, the preparation of hydrolyzed collagen-containing compositions in the form of viscous solutions, gels, creams, pastes, emulsions, balms, and sprays, can be facilitated by the inclusion of water-soluble polymer viscosity builders in amounts ranging from about 0.01 to about 20.0 wt %, from 0.1 to 15% wt, from 0.5 to 10 wt %, or from 1.0 to 5.0 wt %. In some embodiments, the water-soluble polymer viscosity builders useful in the compositions described herein include, but are not limited to, gelatin, carrageenan, xanthan gum, aloe vera gel, and the like.

In some embodiments, the hydrolyzed collagen-containing composition can also include wetting agents, buffers, gelling agents or emulsifiers. Other excipients could include various water-based buffers ranging in pH from 5.0-7.5, surfactants, silicones, polyether copolymers, vegetable and plant fats and oils, hydrophilic and hydrophobic alcohols, vitamins, glycerin, monoglycerides, laurate esters, myristate esters, palmitate esters, and stearate esters.

In some embodiments, the hydrolyzed collagen-containing composition is a dry powder. The hydrolyzed collagen-containing composition may be used in powder form, or the powder may be further processed (e.g., hydrated) into solutions, suspensions, creams, lotions, gels, pastes, emulsions, balms, sprays, foams, aerosols, films, sheets, capsules, tablets or other formulations before application to injured or compromised mucosa.

In some embodiments, one or more biologically active agents may be incorporated into the hydrolyzed collagen-containing composition to provide a medical benefit to a mammalian host. Examples of biologically active agents that can be incorporated into the hydrolyzed collagen-containing composition include, but are not limited to, cells, stem cells, exosomes, amniotic tissue, amniotic cells, mucosal tissue, micronized decellularized tissue, granulated crosslinked bovine tendon collagen and glycosaminoglycans, antibiotics, antiseptics, anti-infective agents, antimicrobial agents, antibacterial agents, antifungal agents, antiviral agents, antiprotozoal agents, sporicidal agents, antiparasitic agents, peripheral neuropathy agents, neuropathic agents, chemotactic agents, analgesic agents, anti-inflammatory agents, anti-allergic agents, anti-hypertension agents, mitomycin-type antibiotics, polyene antifungal agents, decongestants, central nervous system agents, wound healing agents, anti-VEGF agents, anti-tumor agents, escharotic agents, anti-psoriasis agents, anti-diabetic agents, anti-arthritis agents, anti-itching agents, antipruritic agents, anesthetic agents, anti-malarial agents, dermatological agents, anti-arrhythmic agents, anti-convulsants, antiemetic agents, anti-rheumatoid agents, anti-androgenic agents, anthracyclines, anticholinergic agents, anti-aging agents, antihistamines, anti-parasitic agents, hemostatic agents, vasoconstrictors, vasodilators, thrombogenic agents, anti-clotting agents, cardiovascular agents, angina agents, sex hormones, isoflavones, integrin binding sequences, biologically active ligands, cell attachment mediators, immunomodulators, tumor necrosis factor alpha, anti-cancer agents, anti-depressant agents, antitussive agents, anti-neoplastic agents, narcotic antagonists, anti-hypercholesterolemia agents, apoptosis-inducing agents, emollients, alpha-hydroxyl acids, manuka honey, topical retinoids, hormones, tumor-specific antibodies, antisense oligonucleotides, small interfering RNA (siRNA), messenger RNA (mRNA), anti-VEGF RNA aptamer, nucleic acids, DNA, DNA fragments, DNA plasmids, transfection agents, vitamins, essential oils, liposomes, silver nanoparticles, gold nanoparticles, drug-containing nanoparticles, albumin-based nanoparticles, chitosan-containing nanoparticles, polysaccharide-based nanoparticles, dendrimer nanoparticles, phospholipid nanoparticles, iron oxide nanoparticles, bismuth nanoparticles, gadolinium nanoparticles, metallic nanoparticles, ceramic nanoparticles, silica-based nanoparticles, virus-based nanoparticles, virus-like nanoparticles, antibiotic-containing nanoparticles, nitric oxide-containing nanoparticles, nanoshells, nanorods, polymeric micelles, silver salts, zinc salts, quantum dots nanoparticles, polymer-based microparticles, polymer-based microspheres, drug-containing microparticles, drug-containing microspheres, antibiotic-containing microparticles, antibiotic-containing microspheres, antimicrobial microparticles, antimicrobial microspheres, salicylic acid, benzoyl peroxide, 5-tluorouracil, nicotinic acid, nitroglycerin, clonidine, estradiol, testosterone, scopolamine, fentanyl, diclofenac, buprenorphine, bupivacaine, ketoprofen, opioids, cannabinoids, enzymes, enzyme inhibitors, oligopeptides, cyclopeptides, polypeptides, proteins, prodrugs, protease inhibitors, cytokines, hyaluronic acid, chondroitin sulfate, dermatan sulfate, para-sympatholytic agents, chelating agents, lipids, glycolipids, glycoproteins, endocrine hormones, growth hormones, growth factors, differentiation factors, heat shock proteins, immunological response modifiers, polysaccharides, insulin and insulin derivatives, steroids, corticosteroids, and non-steroidal anti-inflammatory drugs or similar materials, in either their salt form or their neutral form, either being inherently hydrophilic or encapsulated within a hydrophilic microparticle or nanoparticle, where applicable, the biologically active agents can be in an (R)-configuration, an (R, S)-configuration, an (S)-configuration, or a combination thereof.

In some embodiments, the hydrolyzed collagen-containing composition may include one or more growth factors. Examples of useful growth factors include, but are not limited to, epidermal growth factor (EGF), transforming growth factor beta (TGF-β), fibroblast growth factor (FGF), vascular endothelial growth factor (VEGF), granulocyte macrophage colony stimulating factor (GM-CSF), platelet-derived growth factor (PDGF), connective tissue growth factor (CTGF), insulin-like growth factor (IGF), keratinocyte growth factor (KGF), interleukin (IL) family, stromal cell derived factor (SDF), heparin binding growth factor (HBGF), nerve growth factor (NGF), brain-derived neurotrophic factor (BDNF), growth differentiation factor (GDF), muscle morphogenic factor (MMF), tumor necrosis factor-alpha (TNFα), and bone morphogenetic proteins (BMP).

In some embodiments, the hydrolyzed collagen-containing composition is hydrated with a fluid. In some embodiments, the mobile phase of the fluid is water, isotonic saline, balanced salt solution, buffer solution, Ringer's solution, cell culture media, stem cell media, serum, plasma, amniotic fluid, Wharton's jelly, nutrient broth, antiseptic solutions, or a combination thereof. In some embodiments, where the fluid is an aqueous media, the aqueous media can have a pH in the range 4 to 8 for mammalian tissue. The pH range will vary depending on targeted mucosal tissue: vaginal 4.0, nasal and anal 5.5-6.5, oral and intestinal 6.3-7.5.

In some embodiments the mobile phase of the fluid is a volatile solvent such as hexamethyldisiloxane, ethanol, and isopropanol.

In another aspect, a method of treating tissue comprising contacting the tissue with a collagen-containing composition as described herein is provided. In some embodiments, the tissue is compromised by trauma, disease, or disease treatment. In some embodiments, the tissue is epithelial tissue. In some embodiments, the tissue is dermal tissue. In some embodiments, the tissue is mucosal tissue.

In some embodiments, the composition is applied topically, via injection, or via ingestion.

In some embodiments, the tissue is compromised tissue. In some embodiments, the tissue is compromised mucosal tissue. The tissue can be compromised physiologically or as side effect of chemotherapy, radiotherapy, or combination thereof, or as a result of infection, disease, cancer, immune response, surgery, cyst, tumor removal, or traumatic injury or remodeling of tissue, such as in skin and wound healing, plastic surgery, cosmetic surgery, reconstructive surgery, coating/sealing of skin replacement products, tendon repair, craniofacial surgery, ophthalmic surgery, cervicofacial rhytidectomy, myocardium repair, cartilage repair, nerve repair, bladder repair, muscle repair, mastopexy, rheumatology, gynecomastia reduction, microsurgery, masking scars or enhancing lips, and the like.

### EXPERIMENTS

The following materials and their abbreviations are used in this experimental section.

| Chemical | Abbreviation | Manufacturer | Lot |
|---|---|---|---|
| Hydrolyzed Collagen | HC | Peptiplus XB, Gelita | 8621390-082322 |
| Kelp Powder | Kelp | XPRS Nutra | CHD-C-A102352 |
| Fucoidan | Fucoidan | Pristine's | FUD240306 |
| Fructo-oligosaccharide | Fructo-OS | Pure Original | 142757 |
| Mannan-oligosaccharide | Manna-OS | Gluten-Free Remedies | 241206 |
| Inulin | Inulin | TCI | NA2VI-IM |
| Carrageenan | Carrageenan | CG-130. CPKelco | SK51142 |

### Example 1: Hydrolyzed Collagen and Low Molecular Weight Polysaccharides

Hydrolyzed collagen was blended with low molecular weight polysaccharides, fructo-oligosaccharide and mannan-oligosaccharide, and evaluated for resulting powder characteristics. The formulations and results are shown in Table 1. In all cases, the resulting powder was free flowing and uniform in color.

TABLE I
Hydrolyzed Collagen and low molecular weight polysaccharides

| HC (g) | Fructo-OS(g) | Mannan-OS (g) | Observations |
|---|---|---|---|
| 3.0008 | 0.0307 | | very light yellow powder, |
| | | | no clumping |
| 3.0003 | | 0.0303 | very light yellow powder, |
| | | | no clumping |
| 3.0007 | 0.3000 | | very light yellow powder, |
| | | | no clumping |
| 3.0009 | | 0.3002 | very light yellow powder, |
| | | | no clumping |
| 3.0006 | 0.0303 | 0.0309 | very light yellow powder, |
| | | | no clumping |

### Example 2: Incorporation of Mucoadhesives

Hydrolyzed collagen (HC) and low molecular weight polysaccharides were mixed with mucoadhesives—kelp, fucoidan and inulin. The formulations and results are shown in Table 2. The resulting powders were free flowing and uniform in color.

TABLE 2
Hydrolyzed collagen, low molecular weight polysaccharides with mucoadhesives

| | Fructo- | Mannan-OS | | Fucoidan | Inulin | |
|---|---|---|---|---|---|---|
| HC (g) | OS (g) | (g) | Kelp (g) | (g) | (g) | Observations |
| 3.0002 | 0.0304 | | 0.0303 | | | light tan powder, no clumping |
| 3.0005 | 0.0303 | | | 0.0306 | | very light yellow powder, no |
| | | | | | | clumping |
| 3.0001 | | 0.0304 | | | 0.0308 | very light yellow powder, no |
| | | | | | | clumping |
| 3.0004 | 0.0305 | 0.0308 | 0.0307 | | 0.0300 | light tan powder, no clumping |
| 3.0001 | 0.0300 | 0.0308 | | 0.0300 | 0.0309 | very light yellow powder, no |
| | | | | | | clumping |

### Example 3: Fibroblast Proliferation and Cytotoxicity

Cytotoxicity and proliferation of different sample concentrations on cell viability were determined using the Alamar Blue assay after a 48-hour exposure.

Fibroblasts were seeded in 96-well plate in Dulbecco's Modified eagle medium (DMEM) with 2% serum, 100 μL of cell suspension (7,500 cells) per well. Plates were incubated at 37° C. under 5% CO₂ for 24 hours to allow the cells to adhere. Treatments were prepared fresh the day of the application and serially diluted in serum free medium (SFM).

The treatments compositions were Hydrolyzed collagen (Cellerate, lot HY024), fructo-oligosaccharides (X003BoNDHT Lot142757), mannan oligosaccharides (Lot 241209), inulin (118-B1, 110041 Lot:NA2VI-IM) and carrageenan. The treatments were prepared using serum free medium (SFM) as the carrier. The concentrations of the treatments was 30 mg/mL, 3 mg/mL, 0.3 mg/mL, and 0.03 mg/mL except for HY024 which was only evaluated at 30 mg/mL. Controls were DMEM with 2% serum and pure SFM.

For treatment, 100 μL of each sample concentration was added to the appropriate wells of the plates and the plates were incubated for 48 hours at 37° C., 5% CO₂ followed by Alamar Blue assay to determine cell proliferation. Alamar Blue reagent was prepared by diluting the original stock 1:10 in phenol red-free cell culture medium. Treatments were removed from the cells and 200 μL of the reagent was added to each well. After 1 hour of incubation, 100 μL of the solution was transferred to a 96-well plate for fluorescence measurement (excitation of 560 nm and emission of 590 nm).

Hydrolyzed collagen, inulin, carrageenan, and fructo-OS retained biocompatibility over the range tested (30 mg/mL-0.03 mg/mL). Mannan-OS and kelp were cytotoxic at 30 mg/mL, but were biocompatible at 0.3 mg/mL and 0.03 mg/mL.

While the above specification contains many specifics, these should not be construed as limitations on the scope of the invention, but rather as examples of preferred embodiments thereof. Many other variations are possible. Accordingly, the scope of this invention should be determined not by the embodiments illustrated, but by the appended claims and their legal equivalents.

What is claimed is:

1. A composition comprising hydrolyzed collagen and low molecular weight polysaccharides.

2. The composition of claim 1, wherein the hydrolyzed collagen is present in an amount ranging from 25 wt % to 99.99 wt % based on the total solids.

3. The composition of claim 1, wherein the hydrolyzed collagen is present in an amount ranging from 80 wt % to 99 wt % based on the total solids.

4. The composition of claim 1, wherein the hydrolyzed collagen has a molecular weight range of up to 12,000 Da.

5. The composition of claim 1, wherein the hydrolyzed collagen is derived from one or more sources selected from a mammalian source, a piscine source, and an avian source.

6. The composition of claim 1, wherein the low molecular weight polysaccharide is present in an amount ranging from 0.01 wt % to 50 wt % based on the total solids.

7. The composition of claim 1, wherein the low molecular weight polysaccharide is present in an amount ranging from 1 to 15 wt % based on the total solids.

8. The composition of claim 1, wherein the low molecular weight polysaccharide has a molecular weight range from 400 Da to 12,000 Da.

9. The composition of claim 1, wherein the low molecular weight polysaccharide is derived from microbial, plant, or animal sources.

10. (canceled)

11. The composition of claim 1, wherein the low molecular weight polysaccharide comprises oligosaccharides.

12. (canceled)

13. The composition of claim 1, further comprising an antimicrobial agent selected from polylysines, biguanides, a low molecular weight synthetic cationic biguanide polymer, chlorhexidine, alexidine, benzalkonium chloride, benzethonium chloride, cetyltrimethylammonium bromide, glycerol mono-laurate, capryl glycol, gentamicin sulfate, iodine, povidone iodine, starch-iodine, neomycin sulfate, polymyxin B, bacitracin, tetracyclines, clindamycin, gentamicin, nitrofurazone, mafenide acetate, copper and its salts, silver nanoparticles, silver sulfadiazine, silver nitrate, terbinafine hydrochloride, miconazole nitrate, ketoconazole, clotrimazole, itraconazole, metronidazole, antimicrobial peptides, polyquaternium-1, polyquatemium-6, polyquaternium-10, salts thereof, or combinations thereof.

14. The composition of claim 1, further comprising a mucoadhesive selected from chitosan, chitosan/β-glycerophosphate, thiolated-chitosan, poly(dimethylaminoethyl methacrylate-co-butyl methacrylate-co-methyl methacrylate), gelatin, polymethylmethacrylate, gellan gum, silica particles, poly(amidoamine), poly(N-vinyl-2-pyrrolidone), poly(vinyl alcohol), poly(2-hydroxyethyl methacrylate), polyethylene oxide block copolymers with polypropylene oxide) (PEO-PPO-PEO), acrylic acid-based polymers, sodium alginate, hydroxypropyl cellulose, carboxymethylcellulose, alginates, pectins, high molecular weight polysaccharides, hydroxyethylcellulose reacted with trimethylammonium substituted epoxide, or combinations thereof.

15. The composition of claim 1, further comprising a biological agent selected from cells, stem cells, exosomes, amniotic tissue, amniotic cells, mucosal tissue, micronized decellularized tissue, granulated crosslinked bovine tendon collagen and glycosaminoglycans, antibiotics, antiseptics, anti-infective agents, antimicrobial agents, antibacterial agents, antifungal agents, antiviral agents, antiprotozoal agents, sporicidal agents, antiparasitic agents, peripheral neuropathy agents, neuropathic agents, chemotactic agents, analgesic agents, anti-inflammatory agents, anti-allergic agents, anti-hypertension agents, mitomycin-type antibiotics, polyene antifungal agents, decongestants, central nervous system agents, wound healing agents, anti-VEGF agents, anti-tumor agents, escharotic agents, anti-psoriasis agents, anti-diabetic agents, anti-arthritis agents, anti-itching agents, antipruritic agents, anesthetic agents, anti-malarial agents, dermatological agents, anti-arrhythmic agents, anti-convulsants, antiemetic agents, anti-rheumatoid agents, anti-androgenic agents, anthracyclines, anticholinergic agents, anti-aging agents, antihistamines, anti-parasitic agents, hemostatic agents, vasoconstrictors, vasodilators, thrombogenic agents, anti-clotting agents, cardiovascular agents, angina agents, sex hormones, isoflavones, integrin binding sequences, biologically active ligands, cell attachment mediators, immunomodulators, tumor necrosis factor alpha, anti-cancer agents, anti-depressant agents, antitussive agents, anti-neoplastic agents, narcotic antagonists, anti-hypercholesterolemia agents, apoptosis-inducing agents, emollients, alpha-hydroxyl acids, manuka honey, topical retinoids, hormones, tumor-specific antibodies, antisense oligonucleotides, small interfering RNA (siRNA), messenger RNA (mRNA), anti-VEGF RNA aptamer, nucleic acids, DNA, DNA fragments, DNA plasmids, transfection agents, vitamins, essential oils, liposomes, silver nanoparticles, gold nanoparticles, drug-containing nanoparticles, albumin-based nanoparticles, chitosan-containing nanoparticles, polysaccharide-based nanoparticles, dendrimer nanoparticles, phospholipid nanoparticles, iron oxide nanoparticles, bismuth nanoparticles, gadolinium nanoparticles, metallic nanoparticles, ceramic nanoparticles, silica-based nanoparticles, virus-based nanoparticles, virus-like nanoparticles, antibiotic-containing nanoparticles, nitric oxide-containing nanoparticles, nanoshells, nanorods, polymeric micelles, silver salts, zinc salts, quantum dots nanoparticles, polymer-based microparticles, polymer-based microspheres, drug-containing microparticles, drug-containing microspheres, antibiotic-containing microparticles, antibiotic-containing microspheres, antimicrobial microparticles, antimicrobial microspheres, salicylic acid, benzoyl peroxide, 5-fluorouracil, nicotinic acid, nitroglycerin, clonidine, estradiol, testosterone, scopolamine, fentanyl, diclofenac, buprenorphine, bupivacaine, ketoprofen, opioids, cannabinoids, enzymes, enzyme inhibitors, oligopeptides, cyclopeptides, polypeptides, proteins, prodrugs, protease inhibitors, cytokines, hyaluronic acid, chondroitin sulfate, dermatan sulfate, para-sympatholytic agents, chelating agents, lipids, glycolipids, glycoproteins, endocrine hormones, growth hormones, growth factors, differentiation factors, heat shock proteins, immunological response modifiers, polysaccharides, insulin and insulin derivatives, steroids, corticosteroids, and non-steroidal anti-inflammatory drugs or similar materials, in either their salt form or their neutral form, either being inherently hydrophilic or encapsulated within a hydrophilic microparticle or nanoparticle, or a combination thereof.

16. The composition of claim 1, wherein the composition comprises a fluid selected from the group consisting of water, isotonic saline, balanced salt solution, buffer solution, Ringer's solution, cell culture media, stem cell media, serum, plasma, amniotic fluid, Wharton's jelly, nutrient broth, antiseptic solutions, and combinations thereof.

17. The composition of claim 1, wherein the composition has a form selected from a powder, a liquid, a gel, a paste, a cream, a suspension, an emulsion, a foam, a lotion, a spray, an aerosol, a film, a sheet, a capsule, or a tablet.

18. A method of treating mammalian tissue comprising contacting the tissue with a composition of claim 1.

19. The method of claim 18, wherein the tissue is mucosal tissue.

20. The method of claim 18, wherein the tissue is compromised by trauma, disease, or disease treatment.
